(19) United States
(12) Patent Application Publication
(10) Pub. No.: US 2012/0232597 A1
(43) Pub. Date: Sep. 13, 2012

(54) FLEXIBLE FIXATION DEVICE

(75) Inventors: Sean Saidha, Basel (CH); Michael White, Basel (CH)

(73) Assignee: SYNTHES USA, LLC, West Chester, PA (US)

(21) Appl. No.: 13/042,641

(22) Filed: Mar. 8, 2011

(51) Int. Cl. A61B 17/86; A61B 17/56

(52) U.S. Cl. 606/305; 606/104; 606/86 R

(57) ABSTRACT

Disclosed are several embodiments directed to a flexible fixation device and method to provide adequate purchase into bone at angles that are awkward for properly engaging inflexible and rigid screws. In one instance, the fixation device is comprised of a head to effect engagement of the device and to acts as an insertion-stop for the device, as well as a helical portion of a suitable cross-sectional geometry to allow for adequate purchase into bone. The helical portion provides fixation as the helical form engages the bone over a relatively large surface area relative to the size of the entry point. The helical portion may be variously configured to resemble, for example, a cork screw, a coiled spring, and so forth. Most variations offer both flexibility (to make angled insertion achievable) while still retaining enough helical-directional rigidity to achieve the desired implantation and fixation.

### BACKGROUND

Vertebral fixation (a.k.a. spinal fixation) is a neurosurgical procedure in which two or more vertebrae are anchored to each other through a synthetic vertebral fixation device. The purpose of the vertebral fixation device is to reduce vertebral mobility in order to mitigate the risk of damage to the spinal cord or spinal nerve roots. A vertebral fixation procedure may be necessary to address instances of vertebral deformity, degenerative vertebral disorders (such as spondylolisthesis), or vertebral fractures.

Today spinal pathologies are being treated more and more often using minimally invasive posterior transpedicular or extrapedicular approaches to emplace spinal implant devices. The devices used to achieve vertebral fixation are often some type of permanent rigid or semi-rigid device made of titanium, titanium alloys, polyetheretherketone or carbon fiber amongst others and may comprise rods, plates, spacers, and various combinations thereof. In order to maintain a spinal implant in place, some form of fixation to bone can be used. In other cases, a vertebral fixation device may be used without the use of supplemental fixation (posterior/plate fixation) in which case, fixation is performed through the vertebral fixation device itself.

The most common means for fixing a device to the bone is to use some form of screw (a.k.a. threaded shaft). Common types of screws used in medical procedures include pedicle screws, facet screws, and plate screws. Stable device fixation can also be achieved using blades, nails, hooks, and other such means, but few (if any) of these approaches can provide the same degree of purchase achievable using a screw-type fixation device.

One of the main drawbacks of screws, however, is that they are rigid and inflexible. Consequently, when a screw (or multiple screws) must be emplaced into a fixed body at an awkward angle with respect to the surgical opening necessary for implanting the spacer or other spinal device, a larger or more extensive opening is necessary to properly engage the screws. Unfortunately, the use of a larger or extended opening may not be consistent with the performance of a “minimally invasive” procedure.

### SUMMARY

Disclosed herein are embodiments of a flexible fixation apparatus (and methods of using same) wherein the flexible fixation apparatus substantially comprises a flexible fixation device comprising a threaded head coupled to a bendable helical structure, and a guide device comprising a threaded fitting corresponding to the threaded head and a helical grooved channel corresponding to the helical structure.

Further disclosed herein embodiments of a flexible fixation device comprising a threaded head and a helical structure couple to the threaded head, wherein the helical structure is bendable upon the application of force.

Further disclosed herein are embodiments of a guide device for a flexible fixation device wherein the guide device comprises an encasement manufactured from a material of greater rigidity than the flexible fixation device, and a shaped channel comprising an ingress and an egress for the flexible fixation device, said channel further comprising a bending feature for bending the flexible fixation device.

### BRIEF DESCRIPTION OF THE DRAWINGS

To facilitate an understanding of and for the purpose of illustrating the present disclosure, exemplary features and implementations are disclosed in the accompanying drawings, it being understood, however, that the present disclosure is not limited to the precise arrangements and instrumentalities shown, and wherein similar reference characters denote similar elements throughout the several views, and wherein:

FIG. 1A is a three-dimensional view of a flexible fixation device representative of several embodiments disclosed herein;

FIG. 1B is a cross-sectional view of the flexible fixation device of FIG. 1A;

FIG. 1C is a cross-sectional view of the flexible fixation device of FIGS. 1A and 1B in an exemplar semi-implanted angled configuration;

FIG. 2 is a three-dimensional view of a flexible fixation device similar to that shown in FIG. 1 representative of certain alternative embodiments disclosed herein;

FIG. 3A is cross-sectional view of an exemplary guide device representative of several embodiments disclosed herein;

FIG. 3B is a three-dimensional view of the exemplary guide device of FIG. 3A representative of several embodiments disclosed herein that utilize two (2) flexible fixation devices;

FIG. 4 is a cross-sectional view of the flexible fixation device of FIGS. 1A, 1B, and 1C fully inserted and engaged in the exemplary guide device of, e.g., FIG. 3A; and

FIG. 5 is an operational flow diagram representing a method for utilizing a guide device together with a flexible fixation device.

### DETAILED DESCRIPTION

In order to allow for minimally invasive insertion of both a spinal device and the fixation device(s) necessary to hold the spinal device in place and/or to provide adequate stability to eliminate the need for supplemental fixation, several embodiments disclosed herein are directed to a flexible fixation device and method that can provide adequate purchase into bone at angles that are awkward for properly engaging inflexible and rigid screws. For certain embodiments, the fixation device is comprised of a head to effect engagement of the device and to acts as an insertion-stop for the device, as well as a helical portion of a suitable cross-sectional geometry to allow for adequate purchase into bone. In such embodiments, the helical portion provides fixation as the helical form engages the bone over a relatively large surface area relative to the size of the entry point. In some embodiments, the helical portion may be configured to resemble a cork screw while in other embodiments the helical portion may be configured to resemble a coiled spring. Regardless of configuration, several embodiments offer both flexibility (to make angled insertion achievable) while retaining enough helical-directional rigidity to achieve the desired implantation and fixation.

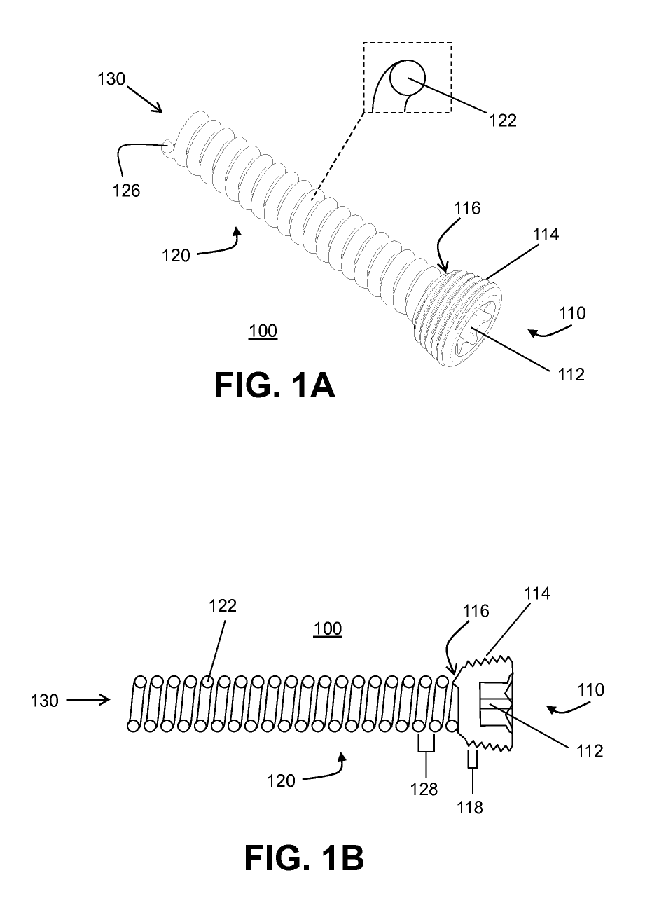

FIG. 1A is a three-dimensional view of a flexible fixation device representative of several embodiments disclosed herein. FIG. 1B is a cross-sectional view of the flexible fixation device of FIG. 1A. FIG. 1C is a cross-sectional view of the flexible fixation device of FIGS. 1A and 1B in an exemplar semi-implanted angled configuration.

In FIGS. 1A, 1B, and 1C, the flexible fixation device 100 is formed to resemble a typical threaded shaft (or screw) and comprises a threaded head 110 featuring a turning tool engagement 112, exterior threads 114, and insertion-stop surface 116, as well as a helical structure 120 comprising a shaft having a suitable crossed-sectional geometry 122 (circular in this exemplary embodiment) coiled around a hollow core 130 running the length of the helical structure 120. Located at the distal end of the helical portion furthest from the head is a sharpened tip 126 to provide efficient cortical bone penetration. In several embodiments, the pitch 118 of the threaded head 110 equals the pitch 128 of the helical portion 120.

The helical structure 120 achieves fixation as its coils gain adequate purchase into the bone over a large area compared to the size of the entry point. In certain embodiments, the helical structure may resemble the screw-portion found on a corkscrew but having enough flexibility to be directionally redirectable (from a first or initial direction to a second or subsequent direction) during insertion (e.g., by a guide device having a stiffer composition) and yet is itself stiffer than the bone it is engaging in order to retain its coiled structure and provide the desired fixation. In some embodiments, the helical structure may be formed such that subsequent helical coils are spaced further apart than the cross-sectional diameter of the coils, while other embodiments may be formed such that the subsequent helical coils are spaced a distance equal to the cross-sectional diameter of the coils. In FIG. 1C, the semi-implanted angled configuration of the flexible fixation device 100 highlights the flexibility of the helical structure 120 at the bend point 150 that can occur anywhere along the length of the helical structure 120 and typical progresses from the tip 126 to a final position more proximal to the threaded head 110.

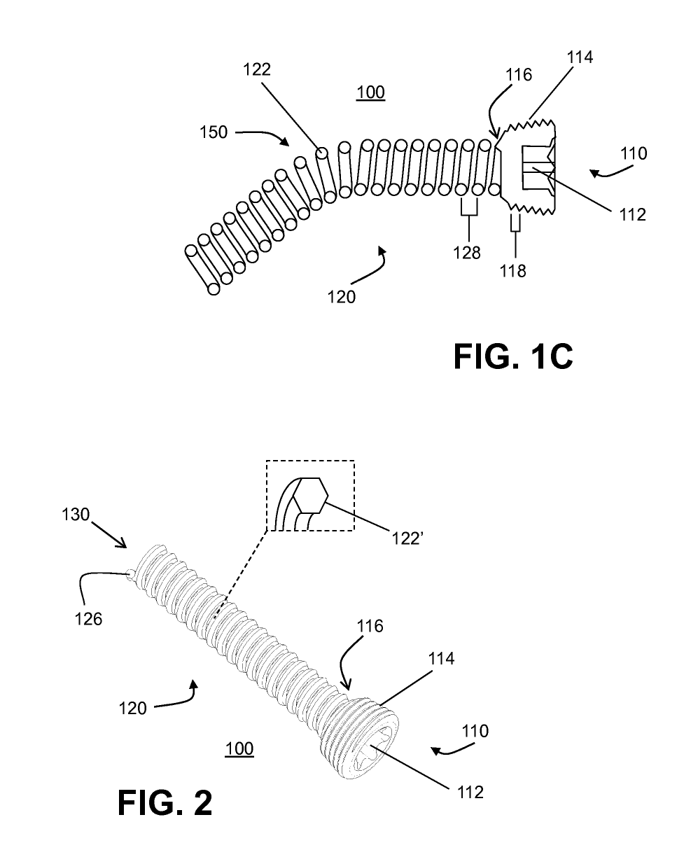

FIG. 2 is a three-dimensional view of a flexible fixation device similar to that shown in FIG. 1 but featuring a helical structure 120 comprising a shaft having hexagonal crossed-sectional geometry 122′. Alternative embodiments employing alternative crossed-sectional geometries are also anticipated. For example, the cross-sectional geometries utilized by some alternative embodiments may include serrations, barbs, or edges to better affix the device in bone or to hinder the withdrawal of the device from bone once implanted.

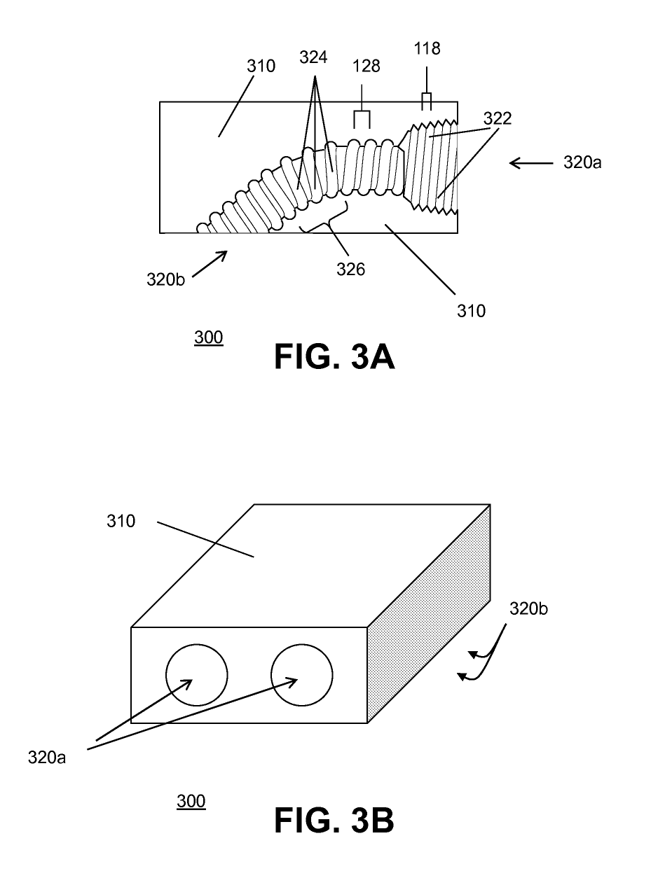

To effect the desired change in angle of the flexible fixation device 100, a guide device may be used. FIG. 3A is cross-sectional view of an exemplary guide device 300 representative of several embodiments disclosed herein. The guide device 300 may be part of another device, for example, an interbody spacer for placement between adjacent vertebrae in the vertebral column. As shown in FIG. 3A, the guide device 300 comprises an encasement 310 manufactured from composition of stiffer and more inflexible qualities than the flexible fixation device 100. The encasement further comprises a shaped channel (extending from 320a to 320b) with an ingress 320a and an egress 320b for a flexible fixation device. The ingress 320a features a threaded fitting 322 matching the pitch 118 of the threaded head 110 of a flexible fixation device 100. Beyond the threaded fitting 322 lies a helical-grooved channel 324 matching the overall diameter of the helical structure 120 of a flexible fixation device 100 and shaped to match the pitch 128 conform to the individual coils comprising the helical structure 120.

The helical-grooved channel 324 further comprises a bending feature 326 that guides the coils of the helical structure 120 into a new direction corresponding to a desired resultant angle different from the direction of travel at the ingress 320a. The bending feature 326 initially distorts the spacing between the coils of the helical structure 120 to make the bend and change the direction of travel, and then the bending feature 326 returns the coils to of the helical structure 120 to roughly their original spacing between the coils before such coils exit the encasement 310 at the egress 320b.

FIG. 3B is a three-dimensional view of the exemplary guide device of FIG. 3A representative of several embodiments disclosed herein that utilize two (2) flexible fixation devices. FIG. 3B illustratively points out that encasement 310 of the guide device 300 may be adapted to receive plural fixation devices. Although two ingress 320a and egress 320b locations are shown, additional ingress 320a and egress locations 320b may be provided.

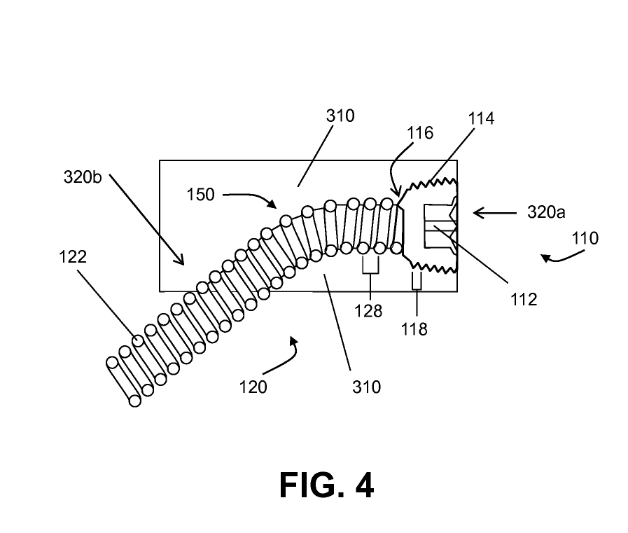

FIG. 4 is a cross-sectional view of the flexible fixation device 100 of FIGS. 1A, 1B, and 1C fully inserted and engaged in the exemplary guide device 300 of, e.g., FIG. 3A.

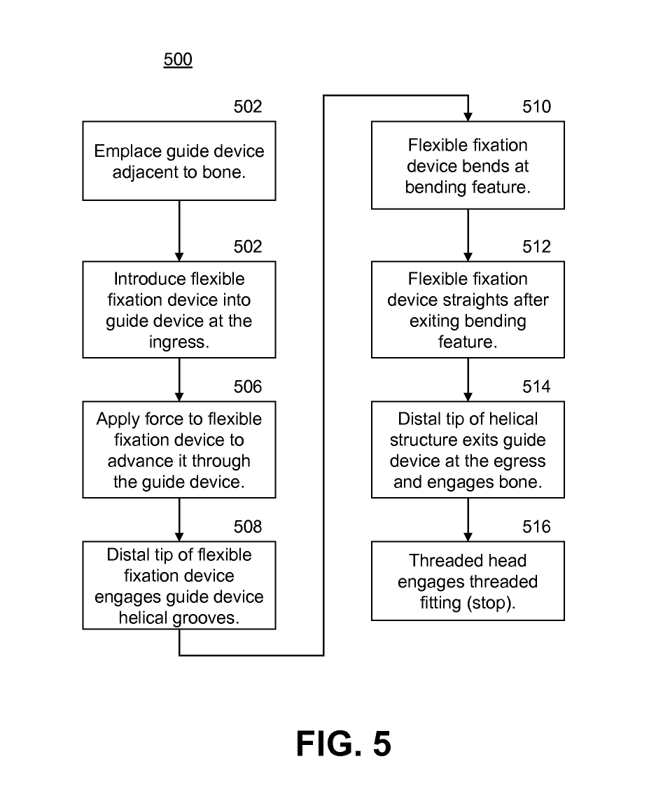

FIG. 5 is an operational flow diagram representing a method 500 for utilizing a guide device 300 together with a flexible fixation device 100. As shown in FIG. 5, at 502 the guide device 300 is emplaced adjacent to bone and ready to be fixed in place. At 504, the flexible fixation device 100 is introduced into the guide device 300 at the ingress 320a. At 506, force (such as a rotational force) is applied to the flexible fixation device 100 to cause it to turn within and advance through the guide device 300. At 508, the distal tip 126 of the fixation device engages a helical groove in the guide device 300. At 510 continued force applied to the flexible fixation device 100 causes the helical structure 120 to bend as it enters the bending feature 324. At 512 continued force applied to the flexible fixation device 100 causes the helical structure 120 to straighten again after passing through the bending feature 324. At 514, and with continued force applied to the flexible fixation device, the distal tip 126 of the helical structure 120 exits the egress and engages bone. At 516, upon further force the threaded head 110 engages into its threaded fitting 322 on the guide device 300.

For certain embodiments, more than one flexible fixation devices 100 may be used to secure a particular guide device 300 in place. Yet other embodiments may utilize a guide wire through the central channel of the guide device 300 (between the ingress 320a and the egress 320b). Yet other embodiments are directed to the utilization of a flexible fixation device in a fixation target other than bone such as, for example, wood, plaster, rock, or any other solid materials. Yet other embodiments are directed to the use of a guide device that is not inserted but operates external to the fixation target.

The subject matter described above is provided by way of illustration only and should not be construed as limiting. Various modifications and changes may be made to the subject matter described herein without following the example embodiments and applications illustrated and described, and without departing from the true spirit and scope of the present invention, which is set forth in the following claims.

What is claimed is:

1. A flexible fixation device comprising:
a threaded head; and
a helical structure couple to the threaded head, wherein an application of a rotational force to the threaded head causes the helical structure to advance in a first direction, and wherein the helical structure is bendable to advance in a second direction upon continued application of the rotational force.

2. The flexible fixation device of claim 1 wherein the threaded head further comprises:
a turning tool engagement; and
an insertion stop surface.

3. The flexible fixation device of claim 1 wherein the helical structure comprises a distal sharpened tip.

4. The flexible fixation device of claim 1 wherein the helical structure comprises a shaft coiled around a hollow core.

5. The flexible fixation device of claim 1 wherein the pitch of the threaded head is equal to the pitch of the helical structure.

6. The flexible fixation device of claim 1 wherein the helical structure comprises a plurality of coils wherein the space between a first coil and a second coil adjacent to the first coil is at least equal to a cross-sectional diameter of the first coil.

7. The flexible fixation device of claim 1 wherein the helical structure comprises a plurality of coils wherein the space between a first coil and a second coil adjacent to the first coil is greater than a cross-sectional diameter of the first coil.

8. The flexible fixation device of claim 1 wherein the helical structure comprises a round cross-sectional geometry.

9. The flexible fixation device of claim 1 wherein the helical structure comprises a hexagonal cross-sectional geometry.

10. The flexible fixation device of claim 1 wherein the helical structure comprises a surface feature for affixing the flexible fixation device in a bone or to hinder the withdrawal of the flexible fixation device from the bone once implanted.

11. The flexible fixation device of claim 10 wherein the surface feature is one from among the following group of surface features: a serration, a barb, and an edge.

12. A guide device for a flexible fixation device, the guide device comprising:
an encasement comprising a material of greater rigidity than the flexible fixation device; and
a shaped channel comprising an ingress and an egress for the flexible fixation device, said channel further comprising a bending feature for bending the flexible fixation device as the flexible fixation device travels between the ingress to the egress.

13. The guide device of claim 12 further comprising a threaded fitting for engaging a threaded head.

14. The guide device of claim 12 wherein the shaped channel is a helical grooved channel for engaging a helical structure.

15. The guide device of claim 12 wherein the bending feature, after bending the flexible fixation device, also unbends the flexible fixation device once a new direction of travel for the flexible fixation device has been achieved.

16. A flexible fixation apparatus comprising:
a flexible fixation device comprising a threaded head coupled to a bendable helical structure; and
a guide device comprising a threaded fitting corresponding to the threaded head and a helical grooved channel corresponding to the helical structure.

17. The flexible fixation apparatus of claim 16 wherein the flexible fixation device is comprised of material more rigid than bone, and wherein the guide device is comprised of material more rigid than the flexible fixation device.

18. The flexible fixation apparatus of claim 16 wherein the pitch of the threaded head is the same as the pitch of the helical structure, and wherein both are the same as the pitch of the threaded fitting and the pitch of the helical grooved channel.

19. The flexible fixation apparatus of claim 16 wherein the apparatus comprises an interbody spacer.

20. The flexible fixation apparatus of claim 16 further comprising at least one additional flexible fixation device.

21. A method for utilizing a flexible fixation device with a guide device, said method comprising:
emplacing the guide device proximate to a fixation target;
introducing the flexible fixation device comprising a helical structure into an ingress in the guide device at a first angle;
applying a rotational force to the flexible fixation device to advance it through the guide device, where advancing the flexible fixation device through the guide device causes the flexible fixation device to change direction; and
engaging the fixation target with the flexible fixation device at an angle different than the first angle.

22. The method of claim 21 wherein the fixation target is a bone.
